(19) United States
(12) Patent Application Publication
(10) Pub. No.: US 2012/0083749 A1
(43) Pub. Date: Apr. 5, 2012

(54) NEEDLE TUBE, MEDICAL INSTRUMENT AND METHOD FOR MANUFACTURING MEDICAL INSTRUMENT

(75) Inventors: HIDEO KAWAMOTO, NAKAKOMA-GUN (JP); TETSUYA OYAUCHI, NAKAKOMA-GUN (JP); KOUICHI AKIYAMA, NAKAKOMA-GUN (JP)

(73) Assignee: TERUMO KABUSHIKI KAISHA, Shibuya-ku (JP)

(21) Appl. No.: 13/325,627

(22) Filed: Dec. 14, 2011

Related U.S. Application Data
(63) Continuation of application No. PCT/EP2010/060984, filed on Jun. 28, 2010.

(30) Foreign Application Priority Data
Jul. 6, 2009 (JP) 2009-159993

(51) Int. Cl. A61B 17/34; B23P 11/00

(52) U.S. Cl. 604/239; 29/428

(57) ABSTRACT

A syringe needle assembly includes a needle tube and a hub. The needle tube has a needle body provided with a needlepoint configured to puncture a living body. To prevent the needle tube from dropping out of a holding member in which the needle tube is held, the needle body also includes an axially press-worked enlarged portion that is continuous in the circumferential direction of the needle body. The hub includes a fitting portion to which the enlarged portion of the needle tube is fitted, and holds the needle tube.

This application is a continuation of International Application No. PCT/JP2010/060984 filed on Jun. 28, 2010, and claims priority to Japanese Application No. 2009-159993 filed on Jul. 6, 2009, the entire content of both of which is incorporated herein by reference.

### TECHNICAL FIELD

The present invention generally relates to a needle tube for puncturing a living body, a medical instrument using the needle tube, and a method for manufacturing the medical instrument.

### BACKGROUND DISCUSSION

Stainless steel is generally used as a material of a syringe needle (needle tube) used when medicinal solution is injected, body fluid is extracted or the like. A needlepoint insertable into a living body is provided at one end of the needle tube. The needle tube as described above is fixed to a holding member such as a hub formed of resin or the like to construct a medical instrument.

One known example of a method of fixing a needle tube to a holding member is disclosed in Japanese Application Publication No. 2002-315740 and is referred to as insert molding. Japanese Application Publication No. 2005-152363 describes enhancing the adhesiveness between the needle tube and the holding member by executing a surface roughening treatment such as an acid treatment, a chemical treatment such as a plasma treatment or the like, a blast treatment or the like on the outer periphery of the needle tube when insert molding is executed. A method using adhesive agent is also known as another method of fixing the needle tube to the holding member.

In the insert molding method described in Japanese Application Publication No. 2002-315740, the surface of the needle tube is smooth and so there is a concern that the needle tube is detached from the holding member. When an attempt is made to enhance the adhesiveness between the needle tube and the holding member by executing the surface treatment on the needle tube as described in Japanese Application Publication No. 2005-152363, the number of working steps is increased, thus reducing the production efficiency.

Even when the needle tube and the holding member are fixed to each other by using adhesive agent, it is necessary to subject the needle tube or the holding member to the surface treatment to enhance the adhesiveness of the adhesive agent.

The needle tube is formed of metal such as stainless or the like, and the holding member is formed of resin such as polypropylene, polyethylene or the like. An adhesive agent which has excellent adhesive characteristics to metal exhibits rather weak adhesive characteristics to resin, and an adhesive agent that has excellent adhesive characteristics to resin exhibits rather weak adhesive characteristics to metal. Therefore, it is necessary to use a surface treatment to enhance the adhesion of the adhesive agent being used on the needle tube or the holding member in accordance with the adhesive agent selected.

### SUMMARY

The disclosure here provides a way of securing the needle in the holding portion to prevent the needle from dropping out of a holding member, without requiring surface treatment on the needle or the holding member.

A medical instrument includes an elongated tubular needle comprised of a needle body possessing a proximal end portion and a distal end portion, with a needlepoint at the distal end portion configured to puncture a living body. The needle body also includes an axially press-worked enlarged portion that is enlarged in a radially outward direction relative to portions of the needle body axially adjoining the axially press-worked enlarged portion. The axially press-worked enlarged portion is located at an intermediate portion of the needle body between the distal end portion and the proximal end portion, and the axially press-worked enlarged portion is continuous in the circumferential direction of the needle body. The medical instrument also includes a holding member having a fitting portion to which the enlarged portion is fitted so that the tubular needle is fixed to the holding member.

Another aspect of the disclosure here involves a method of manufacturing a medical instrument comprising: axially press working a needle body to produce an annular enlarged portion that is enlarged radially outwardly relative to portions of the needle body axially adjoining the enlarged portion and that is continuous in a circumferential direction of the needle body, with the enlarged portion being located at an intermediate portion of the needle body between a distal end portion of the needle body and a proximal end portion of the needle body, and the needle body possessing a needlepoint at the distal end portion of the needle body that is configured to puncture a living body; and fixing the enlarged portion of the needle body relative to a holding portion to form the medical instrument, wherein the enlarged portion is fixed relative to the holding portion to prevent the needle body from axially falling out of the holding member.

According to a further aspect, a needle tube comprises: an elongated needle body possessing a proximal end portion and a distal end portion, with a needlepoint at the distal end portion configured to puncture a living body; and an axially press-worked enlarged portion that is enlarged in a radially outward direction relative to portions of the needle body on axially adjoining opposite sides of the axially press-worked enlarged portion, with the axially press-worked enlarged portion being located at an intermediate portion of the needle body between the distal end portion and the proximal end portion, and the axially press-worked enlarged portion being continuous in a circumferential direction of the needle body.

In the needle tube, the medical instrument and the medical instrument manufacturing method disclosed here, the needle body is subjected to the press working in the axial direction, whereby the annular enlarged portion continuous in the circumferential direction of the needle body is formed on the needle tube. This enlarged portion of the needle tube is fitted to the fitting portion of the holding member, whereby the movement of the needle tube with respect to the holding member is stopped, and the needle tube can be prevented from dropping out of the holding member.

### BRIEF DESCRIPTION OF THE DRAWING FIGURES

Additional features, aspects and details of the needle tube, medical instrument and manufacturing method disclosed here will become more apparent from the following detailed description considered with reference to the accompanying drawing figures which are briefly described below and in which like elements are designated by like reference numerals.

FIG. 1 is a cross-sectional view of a first embodiment of a medical instrument disclosed here.

FIGS. 2A-2C illustrate aspects of a method of manufacturing a enlarged portion in the needle tube according to the first embodiment of the medical instrument disclosed here.

FIG. 3 is a cross-sectional view of the enlarged portion according to the first embodiment of a syringe needle assembly.

FIG. 4 is a cross-sectional view of a second embodiment of a medical instrument disclosed here.

FIG. 5 is a cross-sectional view of a third embodiment of a medical instrument according to the disclosure here.

FIG. 6 is a cross-sectional view of a fourth embodiment of a medical instrument according to the disclosure here.

### DETAILED DESCRIPTION

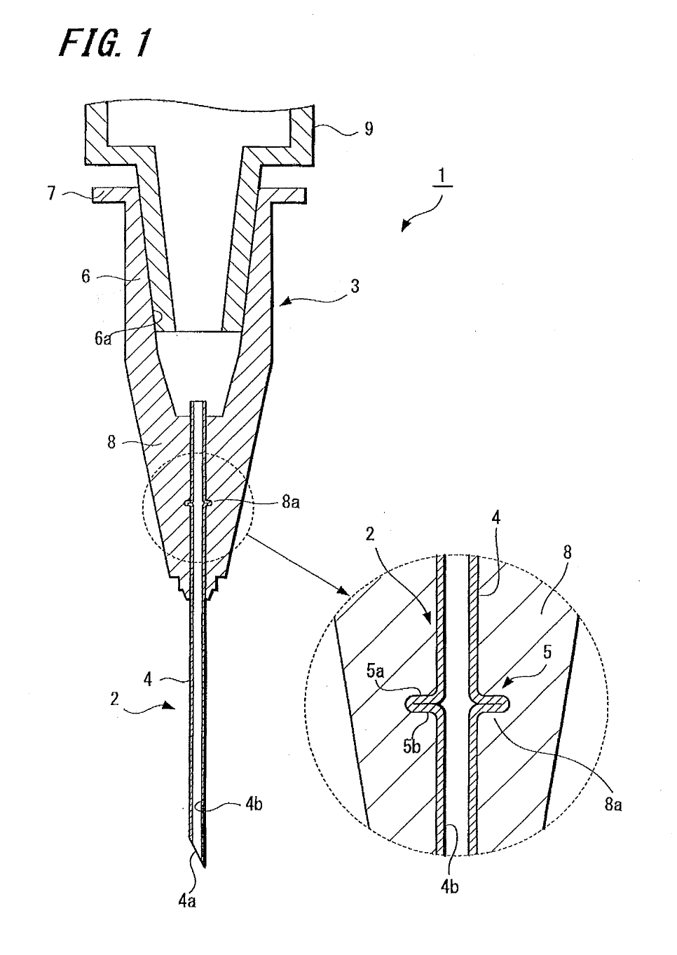

Set forth below is a description of an example of a first embodiment of a medical instrument disclosed here. Referring to FIG. 1, the medical instrument 1 is a syringe needle assembly used when medicinal solution is injected, body fluid is extracted or the like. This medical instrument (hereinafter referred to as “syringe needle assembly”) 1 has a hollow or tubular needle 2 and a hub 3 which holds the tubular needle 2. A syringe 9 is connected to the hub 3 of the syringe needle assembly 1 and constitutes a medical agent injecting device.

The tubular needle 2 has a needle body 4, and an enlarged portion 5 formed integrally in one piece as a part of the needle body 4. The size of the usable needle body 4 ranges from 10 gauge (about 3.4 mm in outer diameter) to 36 gauge (about 0.1 mm in outer diameter), preferably ranging from 14 gauge (about 2.1 mm in outer diameter) to 36 gauge, and more preferably ranging from 28 gauge (about 0.36 mm in outer diameter) to 36 gauge. The free end of the needle body 4 projecting distally beyond the hub 3 is a sharply angled cutting edge face 4a forming the sharp needlepoint at the end of the needle body 4 for puncturing skin during use.

Stainless steel is an example of the material which can be used as the material of the needle body 4. However, the present invention is not limited to this material, and aluminum, aluminum alloy, titan, titan alloy or other metal may also be used. Also, the needle need not be only a straight needle. For example, a double-edged needle or a taper needle having at least a tapered part may also be used. The taper needle may be configured so that the base end portion thereof has a larger diameter than the needlepoint end portion and the intermediate portion thereof is tapered. The cross-sectional shape of the tubular needle 2 is not limited to a circular shape, and it may be a polygonal shape such as a triangle or the like.

The enlarged portion 5 is an axially press-worked portion and is radially outwardly enlarged. The enlarged portion 5 can be provided at any position along the needle body 4. By way of example, the enlarged portion 5 can be located at the intermediate position of the needle body 4 shown in FIG. 1. The enlarged portion 5 is an annular projecting portion continuous in the circumferential direction of the needle body 4 and is formed by press working the needle body 4 in the axial direction. It is preferable that the diameter of the enlarged portion 5 is 1.1 times to three times as large as the outer diameter of the remainder of the needle body 4. A method of manufacturing the tubular needle 2 having this enlarged portion 5 will be described in detail later. One axial end face 5a of the enlarged portion 5 faces the base end side of the needle body, and the other axial end face 5b faces the needlepoint side of the needle body. The end faces 5a and 5b of the enlarged portion 5 are flat faces which are substantially perpendicular to the axial direction of the needle body 4. As illustrated in FIG. 1, the enlarged portion 5 forms a step or shoulder that is not a tapering portion. As mentioned, in this embodiment the enlarged portion 5 can be provided at any position along the needle body 4, though the enlarged portion 5 must be provided at a position allowing it to engage a needle holding portion 8 so that the needle body 4 is fixed to the needle holding portion 8 of a hub 3. The enlarged portion 5 may be provided at any position insofar as it meets the needle holding portion 8.

The hub 3 represents an example of the holding member which holds the tubular needle 2. The hub 3 is formed of synthetic resin such as polycarbonate, polypropylene, polyethylene or the like, and it is formed integrally with the tubular needle 2 by insert molding or welding.

The hub 3 possesses a substantially hollow cylindrical shape, and is comprised of a barrel portion 6, a flange portion 7 and the needle holding portion 8. A barrel hole 6a of the barrel portion 6 communicates with the needle hole or lumen 4b of the tubular needle 2. The syringe 9 is inserted from one end of the barrel portion 6 into the barrel hole 6a, and is detachably connected to the hub 3. The flange portion 7 is provided at the end of the barrel portion 6 from which the syringe 9 is inserted. This flange portion 7 may contact the syringe 9 when the syringe 9 is inserted into the hole 6a.

The needle holding portion 8 of the hub is continuous with the other end of the barrel portion 6 and is located at the end portion of the hub 53 opposite the end which receives the syringe 9. The needle holding portion 8 possesses a substantially circular truncated conical shape so that the outer periphery of the needle holding portion 8 is tapered. The needle holding portion 8 holds an intermediate portion of the tubular needle 2 (the needle body 4) so that the needle 2 (needle body 4) is fixed in place, including axially fixed in place, relative to the holding portion 8. The intermediate portion of the needle holding portion 8 serves as a fitting portion 8a which is brought into close contact with and fitted to the enlarged portion 5 of the tubular needle 2. The enlarged portion 5 of the tubular needle 2 is fitted to the fitting portion 8a, whereby the tubular needle 2 is prevented from moving, including axially moving, with respect to the needle holding portion 8. As a result, the tubular needle 2 can be prevented from dropping out of the hub 3.

The syringe 9 may be configured to be filled with a medical agent, or it may be a pre-filled syringe which is filled with medical agent in advance. Furthermore, the medical agent in the syringe 9 may be a vaccine. However, other medical agents can be used such as a material using high-molecular substance such as cytokine or the like, or hormone. Furthermore, a cutting edge face may be also provided at the other end of the needle body 4, whereby the tubular needle 2 is constructed as a double-edged needle. The other end of the tubular needle 2 may be designed to penetrate through the plug body of the pre-filled syringe and intercommunicate with a vial of medicinal solution or medicine.

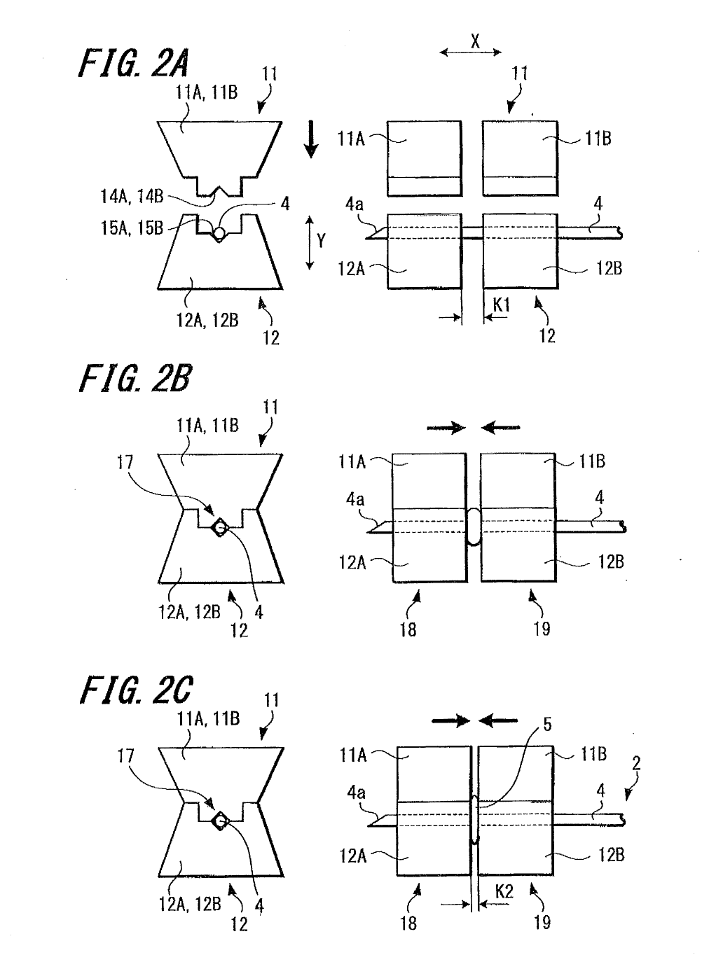

Set forth next, with reference to FIGS. 2A-2C is a description of an example of a method of manufacturing the tubular needle 2 having the enlarged portion 5.

FIG. 2A illustrates an aspect of the method in which the tubular needle 2 is sandwiched by using upper metal molds and lower metal molds, FIG. 2B illustrates the tubular needle 2 being subjected to press working in the axial direction by using the upper metal molds and the lower metal molds, and FIG. 2C illustrates the enlarged portion formed by the press working.

The enlarged portion 5 is formed by subjecting the needle body 4 to press working in the axial direction. A device for performing the press working includes a pair of upper metal molds 11A, 11B and a pair of lower metal molds 12A, 12B.

The two molds forming the pair of upper metal molds 11A, 11B are positioned in juxtaposition or side-by-side relation with each other in a first direction X, and are movable in both the first direction X and a second direction Y perpendicular to the first direction X. The lower surface of the upper metal mold 11A includes a fitting groove 14A, and the lower surface of the other upper metal mold includes a fitting groove 14B. These fitting grooves 14A, 14B extend in the first direction X, and oppose each other in end-to-end relation in the first direction X. The two fitting grooves 14A, 14B are thus aligned in the X direction. Both fitting grooves 14A, 14B possess the same triangular cross-sectional shape. The fitting grooves 14A, 14B of the pair of upper metal molds 11A, 11B are fitted to the circumferential surface of the needle body 4. That is, the fitting grooves 14A, 14B are configured to receive the circumferential surface of the needle body 4.

The two molds constituting the pair of lower metal molds 12A, 12B are positioned in juxtaposition or side-by-side relation with each other in a first direction X as in the case of the fitting grooves 14A, 14B of the pair of upper metal molds 11A, 11B. The upper surface of the lower metal mold 12A faces the lower surface of the upper metal mold 11A in the second direction Y, and the upper surface of the lower metal mold 12B faces the lower surface of the upper metal mold 11B in the second direction Y. The lower metal mold 12A moves in the first direction X together with the upper metal mold 11A, and the lower metal mold 12B moves in the first direction X together with the upper metal mold 11B.

A fitting groove 15A is formed on the upper surface of the lower metal mold 12A, and a fitting groove 15B is formed on the upper surface of the lower metal mold 12B. These fitting grooves 15A 15B extend in the first direction X, and oppose each other in end-to-end relation in the first direction X. The fitting groove 15A of the lower metal mold 12A faces the fitting groove 14A of the upper metal mold 11A in the second direction Y, and the fitting groove 15B of the lower metal mold 12B faces the fitting groove 14B of the upper metal mold 11B in the second direction Y. Both fitting grooves 15A, 15B possess the same triangular cross-sectional shape.

The circumferential surface of the needle body 4 is fitted to the fitting grooves 15A, 15B of the pair of lower metal molds 12A, 12B. That is, the needle body 4 is sandwiched by the upper metal mold 11A and the lower metal mold 12A, and also sandwiched by the upper metal mold 11B and the lower metal mold 12B.

In order to form the enlarged portion 5 integrally with the needle body 4, the axial distance between the upper metal mold 11A (the lower metal mold 12A) and the upper metal mold 11B (the lower metal mold 12B) is initially set to K1 (hereinafter referred to as “distance K1”). Subsequently, the needle body 4 is inserted into the fitting grooves 15A, 15B of the lower metal molds 12A, 12B as shown in FIG. 2A.

Subsequently, the upper metal molds 11A, 11B move in the second direction Y, and are brought into contact with the lower metal molds 12A, 12B, respectively. Accordingly, the needle body 4 is pinched by a first pinching portion 18 comprising the upper metal mold 11A and the lower metal mold 12A, and also pinched by a second pinching portion 19 comprising the upper metal mold 11B and the lower metal mold 12B. The two pinching portions 18, 19 are spaced apart from one another by the distance K1.

At this time, the needle body 4 is engaged with an engaging portion 17 having a rectangular cross-section. This engaging portion 17 is formed by the fitting grooves 14A, 14B which face the fitting grooves 15A, 15B when the upper metal mold 11A and the lower metal mold 12A contact one another, and when the upper metal mold 11B and the lower metal mold 12B contact one another. The needle body 4 is pressed in contact with the four flat faces of the engaging portion 17 so that the needle body 4 is reliably fixed against movement relative to the molds 11A, 11B, 12A, 12B.

Subsequently, the first pinching portion 18 and the second pinching portion 19 are axially moved to approach to each other, and the needle body 4 is subjected to press working in the axial direction thereof as shown in FIG. 2B. Accordingly, an axially compressive force is applied to the needle body 4, and a portion of the needle body 4 located between the first pinching portion 18 and the second pinching portion 19 is swollen or enlarged so as to increase in size in the radial direction so that the enlarged portion possesses a larger circumference than the portion of the needle body immediately adjoining the enlarged portion.

When the interval distance between the first pinching portion 18 and the second pinching portion 19 is equal to K2 (hereinafter referred to as “distance K2”), the movement of the first pinching portion 18 and the second pinching portion 19 is stopped as shown in FIG. 2C. The distance K2 is less than the distance K1 (K1>K2). As a result, the annular enlarged portion 5 is formed so as to be continuous in the circumferential direction of the needle body 4.

The outer diameter and the thickness of the enlarged portion 5 vary in accordance with the outer diameter and thickness of the needle body 4 and the distances K1, K2. Furthermore, the pressing pressure by which the pinching portions 18, 19 are moved towards each other can vary according to the material, outer diameter and thickness of the needle body 4 and the distances K1, K2. That is, the size of the enlarged portion 5 can be adjusted by appropriately selecting the pressing pressure and the distances K1 and K2 for a given tubular needle. For example, when a stainless needle body of 30 gauges (about 0.3 mm in outer diameter and about 0.08 mm in thickness) is used, the distance K1 is set to 0.5 mm, the distance K2 is set to 0.16 mm and press working is executed under a force of about 30 kN (3 tons), and an enlarged portion of about 0.56 mm in outer diameter can be formed.

The position at which the enlarged portion 5 is formed may be set as desired in accordance with the positions of the first pinching portion 18 and the second pinching portion 19 with respect to the needle body 4.

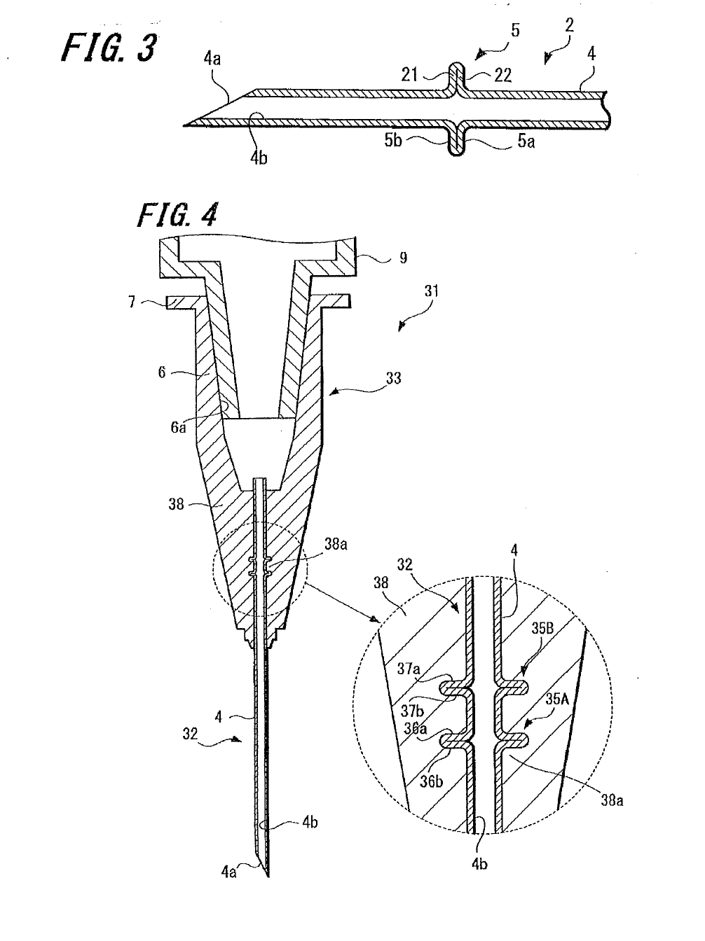

Referring to FIG. 3, the axially press-worked enlarged portion 5 comprises a first enlarged piece 21 raised or enlarged in the radial or circumferential direction of the needle body 4, and a second enlarged piece 22 continuous with the first enlarged piece and folded back on the first enlarged piece 21 so that the first and second enlarged pieces 21, 22 overlap one another in the radial direction. The first enlarged piece 21 is disposed toward one axial side of the needle body 4 to form the end face 5b described above. The second enlarged piece 22 is disposed toward the other axial side (hub side) of the needle body 4 to form the end face 5a described above.

The first enlarged piece 21 and the second enlarged piece 22 overlap each other in the axial direction of the needle body 4. The facing surfaces of the first and second enlarged pieces 21, 22 directly contact one another as shown in the illustrated embodiment of FIG. 3 so that no gap exists between the first enlarged piece 21 and the second enlarged piece 22. The formation of the first enlarged piece 21 and the second enlarged piece 22 does not narrow the needle hole 4b of the needle body 4. Accordingly, even when the enlarged portion 5 is formed, there is no concern that the fluidity of medical agent passing through the needle hole 4b of the needle body 4 is disturbed or restricted.

The following describes a method of manufacturing a syringe needle assembly 1 as a medical instrument. The manufacturing method for making the syringe needle assembly 1 includes a pressing operation and an instrument forming operation.

In the pressing operation, the needle body 4 provided with the needlepoint is subjected to press working in the axial direction to form the annular axially press-worked enlarged portion 5 that is continuous in the circumferential direction of the needle body 4. This pressing operation is described in detail above and is this not repeated again here.

In the instrument forming operation, the hub 3 as the holding member is made to hold the tubular needle 2. In this embodiment, the tubular needle 2 and the hub 3 are formed integrally with each other by insert molding or welding, whereby the enlarged portion 5 of the tubular needle 2 is fitted to the fitting portion 8a of the needle holding portion 8. The fitting position between the enlarged portion 5 and the fitting portion 8a of the needle holding portion 8 may be selected as desired. It is preferably set to a position at which the insert molding can be performed, and it is more preferably set to a position at which the fitting is stronger. As a result, the tubular needle 2 can be inhibited or prevented from dropping out of the hub 3. Accordingly, the tubular needle can be inhibited or prevented from dropping out of the holding member without executing the surface treatment to enhance the adhesiveness between the tubular needle 2 and the hub 3.

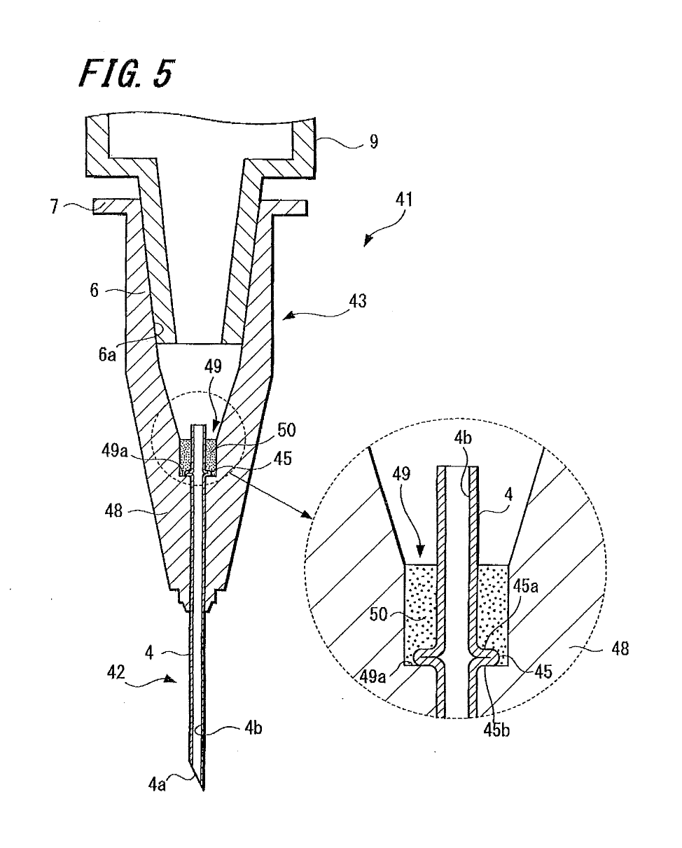

An example of the construction of a second embodiment of a medical instrument according to the disclosure here is set forth with reference to FIG. 4. The medical instrument 31 is a syringe needle assembly used when medicinal solution is injected, body fluid is extracted or the like. This medical instrument (hereinafter referred to as “syringe needle assembly”) 31 has the same construction as the syringe needle assembly 1 of the first embodiment described above and shown in FIG. 1.

The difference of the syringe needle assembly 31 from the syringe needle assembly 3 resides only in two enlarged portions 35A, 35B of the tubular needle 32 and a needle holding portion 38 of the hub 33. Therefore, in the following description, the two enlarged portions 35A and 35B and the needle holding portion 38 will be described. Other parts of the syringe needle assembly 31 which are the same as in the first embodiment described above are identified by the same reference numerals, and a detailed description of such features is omitted.

The two enlarged portions 35A, 35B of the tubular needle 32 are disposed at an intermediate portion of the needle body 4 and are spaced apart from each other at a predetermined interval. The interval or distance between the enlarged portion 35A and the enlarged portion 35B may be set as desired. The minimum distance of the gap between the enlarged portion 35A and the enlarged portion 35B is determined by the length of the fitting grooves 14A, 14B, 15A, 15B of the first pinching portion 18 or the second pinching portion 19.

The two enlarged portions 35A, 35B are formed as annular radially outwardly projecting portions continuous in the circumferential direction of the needle body 4 by executing the press working on the needle body 4 in the axial direction as in the case of the enlarged portion 5 of the first embodiment. This press working comprises, for example, a first press working for forming the enlarged portion 35A and a second press working for forming the enlarged portion 35B.

One axial end face 36a of the enlarged portion 35A faces axially towards the base end side of the needle body, and the other axial end face 36b faces axially toward the needlepoint side. One end face 37a of the enlarged portion 35B faces axially toward the base end side of the needle body, and the other end face 37b faces axially toward the needlepoint side and opposes the end face 36a of the enlarged portion 35A. The end faces 36a, 36b, 37a, 37b of the enlarged portions 35A and 35B are flat faces which are substantially perpendicular to the axial direction of the needle body 4.

The needle holding portion 38 of the hub 33 is continuous with the other end of the barrel portion 6 and is located at the end portion of the hub 33 opposite the end which receives the syringe 9. The needle holding portion 38 possesses a substantially circular truncated conical shape so that the outer periphery of the needle holding portion 38 is tapered. The needle holding portion 38 holds an intermediate portion of the tubular needle 32 (needle body 4) so that the needle holding portion 38 is fixed relative to the tubular needle 32 (needle body 4). An intermediate portion of the needle holding portion 38 serves as a fitting portion 38a which is brought into close contact with and fitted to the enlarged portions 35A, 35B of the tubular needle 32. The fitting of the enlarged portions 35A, 35B of the tubular needle 32 to the fitting portion 38a prevents movement of the tubular needle 32 relatively to the needle holding portion 38. As a result, the tubular needle 32 can be prevented from dropping out of the hub 33.

In the syringe needle assembly 31, the enlarged portions 35A, 35B of the tubular needle 32 are fitted to the fitting portion 38a of the hub 33. That is, two retaining members for the tubular needle 32 are provided. Therefore, the force resisting or preventing the tubular needle 32 from dropping out of the hub 33 is increased, and the drop preventing effect of the tubular needle 32 is enhanced. The tubular needle according to this embodiment may be configured to be provided with three or more enlarged portions.

The method of manufacturing the syringe needle assembly 31 is the same as the method of manufacturing the syringe needle assembly 1 described above, and so the description is not repeated.

An example of the construction of a third embodiment of a medical instrument according to the disclosure here is set forth with reference to FIG. 5. The medical instrument 41 is a syringe needle assembly used when medicinal solution is injected, body fluid is extracted or the like. The medical instrument (hereinafter referred to as “syringe needle assembly”) 41 has the same construction as the syringe needle assembly 1 of the first embodiment described above and shown in FIG. 1.

The difference between this third embodiment of the syringe needle assembly 41 and the syringe needle assembly 1 described above resides only in an enlarged portion 45 of a tubular needle 42 and a needle holding portion 48 of a hub 43. The following description will thus focus primarily on the enlarged portion 45 and the needle holding portion 48. Other parts of the syringe needle assembly 1 which are the same as in the first embodiment described above are identified by the same reference numerals, and a detailed description of such features is not repeated.

The enlarged portion 45 of the tubular needle 42 has the same shape as the enlarged portion 5 according to the first embodiment, and has axial end faces 45a, 45b. The difference between the enlarged portion 45 relative to the enlarged portion 5 resides only in the position at which it is positioned along the tubular needle body 4. The enlarged portion 45 is at a position displaced from the middle portion of the tubular needle body 4 toward the base end side (i.e., toward the side opposite the needlepoint).

As in the case of the hub 3 according to the first embodiment, the hub 43 is formed in a substantially hollow cylindrical shape, and it has a barrel portion 6, a flange portion 7 and a needle holding portion 48. A tubular needle 42 which is provided as a separate body is fixed to the hub 43. Synthetic resin such as polycarbonate, polypropylene, polyethylene or the like may be used as the material of the hub 43, or metal such as stainless, aluminum or the like may be used.

The needle holding portion 48 of the hub 43 is continuous with the other end of the barrel portion 6 and is located at the end portion of the hub 43 opposite the end which receives the syringe 9. The needle holding portion 48 possesses a substantially circular truncated conical shape so that the outer periphery of the needle holding portion 48 is tapered. An intermediate portion of the tubular needle 42 passes through the needle holding portion 48. The needle holding portion 48 includes a fitting recess portion 49 communicating with the barrel hole 6a of the barrel portion 6 (i.e., the barrel hole 6a which receives the syringe 9). The fitting recess portion 49 is positioned distally of the barrel hole 6a. A bottom surface 49a of the fitting recess portion 49 is a flat face or flat surface which is substantially perpendicular to the axial direction of the hub 43. The axial end face 45b of the enlarged portion 45 of the needle tube 42 is fitted to or pressed toward the bottom surface 49a of the fitting recess portion 49. In the illustrated embodiment, the axial end face 45b of the enlarged portion 45 of the needle tube 42 directly contacts the bottom surface 49a of the fitting recess portion 49.

The tubular needle 42 is fixed to the hub 43 by coating the fitting recess portion 49 with adhesive 50 while the tubular needle 42 penetrates through the needle holding portion 48. That is, the adhesive 50 is appropriately introduced into the fitting recess portion 49 with the tubular needle 42 positioned in the needle holding portion 48. The adhesive is limited to the fitting recess portion 49 so that other portions of the needle holding portion 48 surrounding the tubular needle 42 do not contain the adhesive. An adhesive having excellent adhesion characteristics to the hub 43 is used as the adhesive 50. Cyanoacrylate, epoxy resin, photocurable resin or the like may be used as the adhesive 50, though the adhesive 50 may also be generated from other resin materials.

The end face 45b of the enlarged portion 45 is fitted to or brought into contact with the bottom surface 49a of the fitting recess portion 49, whereby the tubular needle 42 is prevented from moving and dropping out of the tip portion of the needle holding portion 48. The adhesive 50 contacts the axially press-worked enlarged portion 45, and also surrounds and directly contacts a portion of the needle body 5 proximal of the axially extending recessed portion 45 as shown in FIG. 5. By coating the fitting recess portion 49 with the adhesive 50, the tubular needle 42 is prevented from moving in all directions, including the direction in which the tubular needle 42 drops out of the tip of the needle holding portion 48. As a result, the tubular needle 42 can be prevented from dropping out of the hub 43.

Even when the adhesion force of the adhesive 50 to the tubular needle 42 is relatively weak, the enlarged portion 45 of the tubular needle 42 is fitted to the hardened adhesive 50, so that the movement of the tubular needle 42 is prevented. Accordingly, the tubular needle 42 can be prevented from dropping out of the hub 43 without subjecting the tubular needle 42 or the hub 43 to the surface treatment for enhancing the adhesiveness of the adhesive agent.

A method of manufacturing the syringe needle assembly 41 as a medical instrument is described next. The method of manufacturing the syringe needle assembly 41 involves execution of a pressing operation and an instrument forming operation.

In the pressing operation, the needle body 4 provided with the needlepoint is subjected to press working in the axial direction to form an annular enlarged portion 45 continuous in the circumferential direction of the needle body 4. This pressing operation is the same as the pressing operation described in detail above with reference to the method of manufacturing the needle tube having the enlarged portion and is thus not described again in detail here.

In the instrument forming operation, the hub 43 forming the holding member is made to hold the needle tube 42. In this embodiment, the intermediate portion of the needle tube 42 is configured and positioned to penetrate through the needle holding portion 48 of the hub 43, and the end face 45b of the enlarged portion 45 is fitted to the bottom surface 49a of the fitting recess portion 49. Subsequently, the fitting recess portion 49 of the hub 43 is coated with the adhesive 50, and the needle tube 42 is thus fitted to the needle holding portion 48.

Accordingly, the movement of the needle tube 42 with respect to the needle holding portion 48 is inhibited or prevented. Therefore, the needle tube 42 can be inhibited or prevented from dropping out of the hub 43 without subjecting the needle tube 42 or the hub 43 to the surface treatment for enhancing the adhesiveness of the adhesive agent.

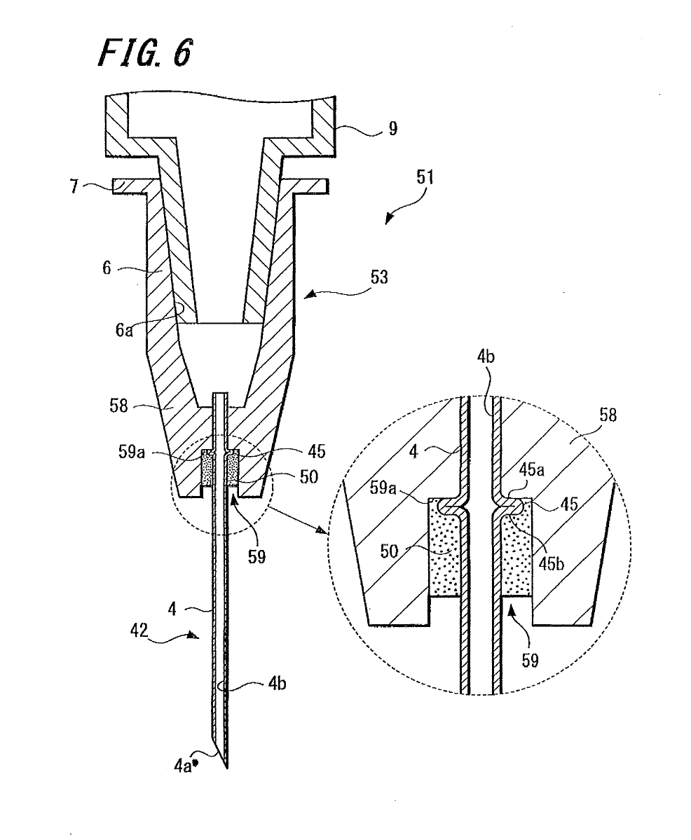

An example of the construction of a fourth embodiment of a medical instrument according to the disclosure here is set forth with reference to FIG. 6. The medical instrument 51 is a syringe needle assembly used when medicinal solution is injected, body fluid is extracted or the like. The medical instrument (hereinafter referred to as “syringe needle assembly”) 51 has a construction similar to the syringe needle assembly 41 of the third embodiment described above and shown in FIG. 5.

The difference between the syringe needle assembly 51 of this fourth embodiment and the syringe needle assembly 41 described earlier resides only in the needle holding portion 58 of the hub 53. Therefore, the description which follows focuses primarily on the needle holding portion 58. Other parts of the syringe needle assembly 41 which are the same as in the third embodiment described above are identified by the same reference numerals, and a detailed description of such features is not repeated.

The hub 53 has the barrel portion 6, the flange portion 7 and the needle holding portion 58. The needle tube 42 which is formed as a separate body from the hub 53 is fixed to the hub 53. Synthetic resin such as polycarbonate, polypropylene, polyethylene or the like may be used as the material of the hub 53, and metal such as stainless, aluminum or the like may also be used.

The needle holding portion 58 of the hub 53 is continuous with the other end of the barrel portion 6 and is located at the end portion of the hub 53 opposite the end which receives the syringe 9. The needle holding portion 58 possesses a substantially circular truncated conical shape so that the outer periphery of the needle holding portion 58 is tapered. An intermediate portion of the needle tube 42 passes through the needle holding portion 58. The needle holding portion 58 is provided with a fitting recess portion 59 which is opens axially toward the tip end side of the needle holding portion 58 (i.e., the fitting recess portion 59 opens toward the distal direction). The bottom surface 59a of the fitting recess portion 59 is a flat face or flat surface which is substantially perpendicular to the axial direction of the hub 53. The axial end face 45a of the enlarged portion 45 of the needle tube 42 is fitted to or pressed toward the bottom surface 59a of the fitting recess portion 59. In the illustrated embodiment, the axial end face 45a of the enlarged portion 45 of the needle tube 42 directly contacts the bottom surface 59a of the fitting recess portion 59.

The needle tube 42 is fixed to the hub 53 by coating the fitting recess portion 59 with the adhesive 50 under the state that the needle tube 42 penetrates through the needle holding portion 58. That is, the adhesive is appropriately introduced into the fitting recess portion 59 with the tubular needle 42 positioned in the needle holding portion 58. At this time, the end face 45a of the enlarged portion 45 is fitted to or in direct contact with the bottom surface 59a of the fitting recess portion 59, whereby the needle tube 42 is prevented or inhibited from moving so as to drop out of the rear end (the barrel portion 6 side) of the needle holding portion 58. Accordingly, the needle tube 42 can be surely prevented or inhibited from dropping (releasing) out of the hub 53 due to resistance applied to the needle tube when the needle tube 42 is used to puncture skin or the like. The adhesive 50 contacts the axially press-worked enlarged portion 45, and also surrounds and directly contacts a portion of the needle body 5 distal of the axially extending recessed portion 45 as shown in FIG. 6.

Next, a method of manufacturing the syringe needle assembly 51 as a medical instrument will be described. The method of manufacturing the syringe needle assembly 51 involves execution of a pressing operation and an instrument forming operation. In the pressing operation, the needle body 4 provided with the needlepoint is subjected to press working in the axial direction to form an annular enlarged portion 45 continuous in the circumferential direction of the needle body 4. This pressing operation is the same as the pressing operation described in detail above with reference to the method of manufacturing the needle tube having the enlarged portion and is thus not described again in detail here.

In the instrument forming operation, the hub 53 as a holding member is made to hold the needle tube 42. In this embodiment, the end portion of the needle tube 42 opposite the needlepoint 4a is inserted from the tip side (distal end) of the needle holding portion 58, and the axial end face 45a of the enlarged portion 45 is fitted to or brought into contact with the bottom surface 59a of the fitting recess portion 59. Subsequently, the fitting recess portion 59 of the hub 53 is coated or filled with the adhesive 50 to fix the needle tube 42 to the needle holding portion 58.

Accordingly, the movement of the needle tube 2 with respect to the needle holding portion 58 is inhibited or prevented. At this time, even when the adhesion force of the adhesive 50 to the needle tube 42 is weak, the enlarged portion 45 of the needle tube 42 is fitted to the hardened adhesive 50, and thus the movement of the needle tube 42 is prevented. Accordingly, the needle tube 42 can be inhibited or prevented from dropping out of the hub 53 without subjecting the needle tube 42 or the hub 53 to the surface treatment for enhancing the adhesiveness of the adhesive agent.

The description above describes embodiments of a needle tube, medical instrument and medical instrument manufacturing method. But the needle tube, the medical instrument and the medical instrument manufacturing method are not limited to the disclosure above. For example, the syringe needle assembly is not limited to the general syringe needle illustrated in the drawing figures and described above, but applies also to a winged intravenous injection needle (winged needle), an indwelling needle, a needle-attached pre-filled syringe pre-filled with medicinal solution, an insulin syringe needle (pen needle) or the like. Furthermore, the medical instrument is not limited to a syringe needle. For example, the medical instrument may be a blood sampling needle.

In the above embodiments, the syringe needle assembly in which the needle tube is fixed to the hub is described as an example of the medical instrument. However, the medical instrument of the present invention is not limited in this regard. It is sufficient that the medical instrument has a needle tube and a holding member for holding the needle tube. For example, the medical instrument may have a needle tube and a syringe for holding the needle tube.

The detailed description above describes features and aspects of embodiments of an needle tube, the medical instrument and the medical instrument manufacturing method disclosed by way of example. The invention is not limited, however, to the precise embodiments and variations described. Changes, modifications and equivalents can be employed by one skilled in the art without departing from the spirit and scope of the invention as defined in the appended claims. It is expressly intended that all such changes, modifications and equivalents which fall within the scope of the claims are embraced by the claims.

What is claimed is:

1. A medical instrument comprising:
an elongated tubular needle comprised of a needle body possessing a proximal end portion and a distal end portion, with a needlepoint at the distal end portion configured to puncture a living body;
the needle body also including an axially press-worked enlarged portion that is enlarged in a radially outward direction relative to portions of the needle body axially adjoining the axially press-worked enlarged portion, the axially press-worked enlarged portion being located at an intermediate portion of the needle body between the distal end portion and the proximal end portion, the axially press-worked enlarged portion being continuous in a circumferential direction of the needle body; and
a holding member having a fitting portion to which the enlarged portion is fitted so that the tubular needle is fixed to the holding member.

2. The medical instrument according to claim 1, wherein the needle body possesses an outer diameter of 10 gauge-36 gauge at the portions of the needle body axially adjoining the enlarged portion.

3. The medical instrument according to claim 1, wherein the enlarged portion comprises a first enlarged piece on one axial side of the enlarged portion and a second enlarged piece continuous with the first enlarged piece on an opposite axial side of the enlarged portion from the first enlarged piece, the first and second enlarged pieces radially overlapping one another.

4. The medical instrument according to claim 3, wherein the first enlarged piece and the second enlarged piece each have facing surfaces which contact one another.

5. The medical instrument according to claim 1, wherein one axial side of the axially press-worked enlarged portion is a flat surface facing towards the needlepoint, the flat surface being substantially perpendicular to an axial direction of the needle body.

6. The medical instrument according to claim 1, wherein the needle body includes a plurality of spaced-apart axially press-worked enlarged portions each enlarged in the radially outward direction relative to the portions of the needle body axially adjoining each respective enlarged portion, each of the axially press-worked enlarged portions being continuous in a circumferential direction of the needle body.

7. The medical instrument according to claim 1, wherein the fitting portion of the holding member includes an axially extending recessed portion which is radially enlarged relative to a remainder of the fitting portion, and further comprising an adhesive in the axially extending recessed portion that is in contact with the axially press-worked enlarged portion.

8. The medical instrument according to claim 7, wherein the adhesive surrounds and directly contacts a portion of the needle body located proximally of the axially press-worked enlarged portion.

9. The medical instrument according to claim 7, wherein the adhesive surrounds and directly a portion of the needle body located distally of the axially press-worked enlarged portion.

10. The medical instrument according to claim 1, wherein the axially press-worked enlarged portion is located closer to the proximal end portion of the needle body than the distal end portion of the axial body.

11. A method of manufacturing a medical instrument comprising:
axially press working a needle body to produce an annular enlarged portion that is enlarged radially outwardly relative to portions of the needle body axially adjoining the enlarged portion and that is continuous in a circumferential direction of the needle body, the enlarged portion being located at an intermediate portion of the needle body between a distal end portion of the needle body and a proximal end portion of the needle body, the needle body possessing a needlepoint at the distal end portion of the needle body that is configured to puncture a living body; and
fixing the enlarged portion of the needle body relative to a holding portion to form the medical instrument, the enlarged portion being fixed relative to the holding portion to prevent the needle body from axially falling out of the holding member.

12. The medical instrument manufacturing method according to claim 11, wherein the axially press working of the needle body comprises pinching the needle body at a first pinching portion and a second pinching portion that are spaced from each other by an axial distance forming a first predetermined distance, and axially moving at least one of the first pinching portion and the second pinching portion to reduce the axial distance between first pinching portion and the second pinching portion to less than the predetermined distance.

13. The medical instrument manufacturing method according to claim 11, wherein the fixing of the enlarged portion of the needle body relative to the holding portion forms the holding member and the needle tube integrally with each other.

14. The medical instrument manufacturing method according to claim 11, wherein the fixing of the enlarged portion of the needle body relative to the holding portion includes using an adhesive to fix the needle tube relative to the holding member.

15. The medical instrument manufacturing method according to claim 11, wherein the axially press working of the needle body comprises pinching one intermediate portion of the needle body between two first molds positioned in opposing relation to each, pinching an other intermediate portion of the needle body between two second molds positioned in opposing relation to each other, with the two first molds being spaced apart from the two second molds so that an axially extending space exists between the two first molds and the two second molds, the method further comprising moving the two first molds axially toward the second two molds to reduce the axially extending space and apply an axially compressive force to the needle body to produce the enlarged portion.

16. A needle tube comprising:
an elongated needle body possessing a proximal end portion and a distal end portion, with a needlepoint at the distal end portion configured to puncture a living body; and
an axially press-worked enlarged portion that is enlarged in a radially outward direction relative to portions of the needle body on axially adjoining opposite sides of the axially press-worked enlarged portion, the axially press-worked enlarged portion being located at an intermediate portion of the needle body between the distal end portion and the proximal end portion, the axially press-worked enlarged portion being continuous in a circumferential direction of the needle body.

17. The needle tube according to claim 16, wherein the axially press-worked enlarged portion comprises a first enlarged piece on one axial side of the axially press-worked enlarged portion and a second enlarged piece continuous with the first enlarged piece on an opposite axial side of the axially press-worked enlarged portion from the first enlarged piece, the first and second enlarged pieces radially overlapping one another.

18. The needle tube according to claim 17, wherein the first enlarged piece and the second enlarged piece each have facing surfaces which contact one another.

19. The needle tube according to claim 16, wherein one axial side of the axially press-worked enlarged portion is a flat surface facing towards the needlepoint, the flat surface being substantially perpendicular to an axial direction of the needle body.

20. The needle tube according to claim 16, wherein the needle body includes a plurality of spaced-apart axially press-worked enlarged portions each enlarged in the radially outward direction relative to the portions of the needle body axially adjoining each respective enlarged portion, each of the axially press-worked enlarged portions being continuous in a circumferential direction of the needle body.
